(19) United States
(12) Patent Application Publication
(10) Pub. No.: US 2012/0178799 A1
(43) Pub. Date: Jul. 12, 2012

(54) ENERGY BEVERAGE WITH CREATINE AND CAFFEINE COMBINATION

(76) Inventor: Terence O'Neil, Corona Del Mar, CA (US)

(21) Appl. No.: 13/344,774

(22) Filed: Jan. 6, 2012

Related U.S. Application Data
(60) Provisional application No. 61/430,809, filed on Jan. 7, 2011.

(51) Int. Cl. A61K 31/335; A23L 1/302; C12G 3/00; A61P 7/06; A61P 1/16; A61K 31/195; A61P 3/10; A23L 2/38; A23L 1/305

(52) U.S. Cl. 514/452; 426/590; 426/72; 426/592; 514/561

(57) ABSTRACT

The present invention is an energy drink that included both creatine and caffeine, which provides superior energy, concentration and performance over other energy beverages/liquids. The invention can include other vitamins and beneficial additives, but is intended to be unflavored and unsweetened.

### CROSS-REFERENCES TO RELATED APPLICATIONS

This application claims priority from U.S. Provisional Application No. 61/430,809, filed Jan. 7, 2011 incorporated by reference in its entirety.

### BACKGROUND

There is a bounty of beverages that have been introduced into the market in recent times that can generally be referred to as “energy drinks ” According to the New York Times, nearly 200 new energy drinks have hit store shelves in the last year alone, based on a report by the market research firm ACNielsen. Led by such brands as Red Bull™, Rockstar™ and Monster™, energy drinks are a $3.7 billion industry whose revenues have increased by 51 percent in the past year alone. Typically sugar based, most of these energy drinks profess to offer the consumer a burst of energy that help to get a person through the day. The response to these energy drinks show that there is an interest in this market, and it should continue to grow in the future.

Most of the energy drinks in the market rely on either caffeine or sugar, or both, to provide a pick-me-up. The sudden influx of concentrated sugar provides the body with a rush of energy that can provide short term relief for tiredness or sleepiness. However, large doses of sugar is not a healthy solution to the problem, and many doctors have come out against such high sugar drinks as unhealthy to young adults. The present invention is a healthier alternative to the offerings currently on the market.

### SUMMARY OF THE INVENTION

The present invention is an energy drink with a unique combination of creatine and caffeine, which provides superior energy, concentration and performance over other energy beverages/liquids. While other beverages rely on added flavoring and sweeteners to appeal to a younger market, whose cravings for sweetened drinks helps drive the industry, the present invention is directed to a potential different market. That is, the present invention provides superior energy, mental focus, and performance benefits, and is unique in both constitution and its target consumer. This invention has no artificial flavoring or sweeteners, although the invention can be mixed into any other beverage to make one's own energy drink, while maintaining the beverage's basic taste.

In addition, this drink contains the unique combination of creatine and caffeine, which heretofore has not been combined in a beverage/liquid or other substance in order to promote energy and improve metal thought. Creatine is a naturally existing chemical in the body that, when consumed before a workout, helps the body to accelerate the production of ATP faster than one's body can make. ATP, or adenosine triphosphate, provides energy to the muscles. The increased production of ATP is reported to lead to improved energy and concentration. The improved concentration is a result of the fact that the brain uses a large amount of ATP quickly. Therefore, if one can produce ATP faster, one can as a result have improved concentration. The addition of caffeine to the formula adds a stimulant, which has also been shown be improve energy, and mental concentration. The combination of creatine, or creatine derivatives, is an ideal combination to improve mental awareness. The other purpose of this invention is to additionally provide vitamins, in order to replenish the body, particularly after alcohol consumption, lesson the symptoms of alcohol consumption, and provide the body with a quick supply of energy. Also the product has the unique advantage of providing a vitamin shot that has specifically been designed as a vitamin ‘mixer’ for alcoholic drinks

Currently the art lacks an energy drink that has the unique combination of creatine and caffeine. In addition, it is unique as it is unflavored, unsweetened, and suitable as an energy mixer. The creatine-caffeine combination highlights its uniqueness, coupled with the fact that it is unflavored, and unsweetened. In addition, in a preferred embodiment the formulation is a unique combination of vitamins, creatine, silymarin, taurine, glycine, and caffeine.

### DETAILED DESCRIPTION OF THE PREFERRED EMBODIMENTS

The present invention is a creatine and caffeine based beverage that provides short term energy and improves mental acuity and awareness. The first ingredient is creatine, a natural component found primarily in the body's skeletal muscle. Sources of ingested creatine include such proteins as meat and fish. After ingestion, creatine is transported to muscles to increase their energy levels by increasing the availability of ATP. The increased ATP provides an extra kick during repeated bouts of intense exercise which helps the body build more muscle. Other benefits of creatine include the following:

1. Creatine rebuilds ATP. ATP, or more technically adenosine triphosphate, is the chemical in the body that powers a body's muscles. When exercising, ATP helps the body's muscles contract. With each muscle contraction, the amount of ATP in a person's system decreases until it is depleted and the muscle becomes fatigued. The only way to replenish ATP in your muscles is with creatine.

Creatine increases the body's ability to store glycogen. Muscles use glycogen to fuel the anabolic process, and an adequate supply of glycogen is needed to help muscles recover after an intense workout. This enables muscles to grow bigger and faster.

Creatine supplements can be used to augment the need for creatine in the body if one is not getting enough though one's diet. Creatine is mainly found in red meats, like steak, so vegetarians or anyone who doesn't eat a lot of creatine-hearty foods may benefit from the use of supplements. Glycogen, a carb, is often achieved by carb-loading (i.e., eating a lot of pasta or bread), so taking creatine supplements will reduce the need to take in large amounts of carbohydrates when training or the like.

Creatine has been observed to increases cardiovascular activity as well. Increased creatine has been reported to increase anabolic workouts, and positively influence cardiovascular exercise as well. Creatine helps increase the amount of aerobic activity one can do before getting winded, so one will be able to exercise longer and more intensely.

The other main ingredient besides creatine is caffeine, a well-known stimulant. Caffeine had long been on the list of don'ts for people hoping to lead a healthy lifestyle. Recent studies show that caffeine offers some significant medical benefits. For example, studies have shown that regular coffee drinkers were 80 percent less likely to develop Parkinson's disease. Further, those who consumed two cups of coffee a day reduced a subjects' risk for colon cancer by 20 percent, and also caused an 80 percent drop in the odds of developing cirrhosis while cutting the risk of developing gallstones in half.

Studies have also suggested that caffeine is beneficial in treating asthma, stopping headaches, boosting mood and even preventing cavities. Some of these findings may have something to do with other healthful properties of the coffee bean, but most can be linked to caffeine directly. Researchers are even developing drugs for Parkinson's disease containing caffeine derivatives. More research is uncovering potential benefits from this commonly consumed drug. A study by the Byrd Alzheimer's Institute in Tampa, Fla., showed that lab mice injected with caffeine were protected against developing Alzheimer's disease. The injections even helped reduce symptoms in those that had the disease. The findings lead doctors to believe that up to five cups of coffee a day could have the same positive effect on humans. Finally, a 2007 study at Rutgers University suggested that regular exercise combined with daily doses of caffeine could increase the destruction of precancerous skin cells in mice. Once again, the findings have not yet been tested on humans, but the indication is that it will have similar effects.

The present inventor has found that the symbiotic relationship between the caffeine and the creatine provide unique benefits that surpass the individual benefits of each drug individually. Combined in this new beverage which is the subject of the present invention, the combination of creatine and caffeine improve energy and mental awareness without some of the drawbacks of the individual constituents.

Other constituents of the present invention include vitamin B1. Vitamin B1 (thiamine) is an important vitamin required by the human body. It can be found in meat, green leaves, and vegetables and is essential for the breakdown of fat and protein. It also keeps the mucous membranes healthy. The top five health benefits of vitamin B1 are:

1. Energy production: Vitamin B1 is responsible for converting sugar into energy. The vitamin acts as a co-enzyme in oxidizing sugar to produce energy for the smooth functioning of the body organs, especially the heart, brain, lungs, and kidneys.

2. Cardiovascular functions: This vitamin is responsible for the production of acetylcholine, a neurotransmitter that relays messages to the muscles and nerves. A deficiency of vitamin B1 leads to a decrease in the neurotransmitter and causes irregular heartbeat. Severe deficiency can cause congestive heart failure.

3. Eye health benefits: Essential fatty acids Omega-3 and Omega-6 together with vitamin B1 help ensure eye health and prevent the formation of cataracts.

4. Improves brain function: It ensures smooth functioning of the brain and helps improve memory and concentration. Vitamin B1 helps relieve stress and also helps strengthen the nerves. The vitamin is used to reduce the progression of multiple sclerosis, Alzheimer's disease, cirrhosis, and other infections.

5. Development of myelin sheaths: Myelin sheaths are the protective covering of the nerves. Deficiency of vitamin B1 results in weakening of the sheaths. Adequate intake of vitamin B1 ensures the development of myelin sheaths and aids nerve functioning.

Another ingredient of a preferred embodiment of the present invention is Vitamin B6. Vitamin B6 is a vital nutrient which is responsible for the proper function of sixty enzymes within the human body. Vitamin B6 contributes in the manufacturing of serotonin, which is a chemical within the brain. Vitamin B6 also plays a major part in the production of hemoglobin and cells within our immune system. Vitamin B6 also is involved in the process of breaking down carbohydrates, and processing it to be turned into energy for the body's daily activities. A decrease in vitamin B6 can also contribute to a poor nervous system. Vitamin B6 is also a helper in regulating estrogen and progesterone.

Another ingredient of the preferred embodiment is Vitamin B12. Vitamin B12 is a water-soluble vitamin that keeps your nerves and red blood cells healthy. It is responsible for the smooth functioning of several critical body processes. Strict vegetarians, heavy drinkers and smokers, pregnant and breast-feeding women, and the elderly usually require vitamin B 12 supplements. Sometimes the digestive system is not able to absorb this vitamin well. This can happen when a person has pernicious anemia, celiac disease, Crohn's disease, bacteria growth in the small intestine, or a parasite. People suffering from these conditions may need vitamin B12 supplements. A deficiency in vitamin B12 can result in a host of illnesses like anemia, fatigue, weakness, constipation, loss of appetite, weight loss, depression, poor memory, soreness of the mouth, asthma, vision problems, and a low sperm count.

The top 5 health benefits of vitamin B 12 are:

1. It is needed to convert carbohydrates into glucose in the body, thus leading to energy production and a decrease in fatigue and lethargy in the body.

2. It helps in healthy regulation of the nervous system, reducing depression, stress, and brain shrinkage.

3. It helps maintain a healthy digestive system. Vitamin B12 also protects against heart disease by curbing and improving unhealthy cholesterol levels, protecting against stroke, and high blood pressure.

4. It is essential for healthy skin, hair, and nails. It helps in cell reproduction and constant renewal of the skin.

5. Vitamin B 12 helps protect against cancers including breast, colon, lung, and prostrate cancer.

Yet another ingredient of the present invention is folic acid. This folic acid has demonstrated a protective effect against cardiovascular diseases, some forms of cancer, and various neurological impairments including depression. Scientists believe that many people may benefit from folic acid doses far exceeding what is found in today's dietary supplements. Folic acid is an important addition to the disease-prevention arsenal, particularly against cardiovascular disease, neuropsychiatric disorders, and certain cancers. Furthermore, while most folic acid studies have focused on heart health, some recent findings suggest that folic acid either has antidepressant properties or can act as an augmenting mediator for standard antidepressant treatment.

Taurine is another ingredient in the present invention. Taurine is a non-essential amino acid produced by the body through the synthesis of two other amino acids, namely methionine and cysteine. It is an important component of bile acids, which are used to absorb fats and fat-soluble vitamins. It also regulates heartbeat; maintains the stability of cell membranes; transports calcium in and out of cells; and regulates the activity of brain cells. It is also a potent antioxidant. Taurine is believed to play a role in treating a number of conditions, including congestive heart failure, high blood pressure, diabetes, and retinal damage. Normally our bodies manufacture taurine rather than obtain it from our diet. It is produced by a combination of cysteine, methionine and vitamin C, but low amounts of these substances can in turn lead to taurine deficiency.

Another ingredient of the preferred embodiment of the present invention is glycine. Glycine is a non-essential amino acid that helps to create muscle tissue and convert glucose into energy. It is also essential to maintaining healthy central nervous and digestive systems, and has recently been shown to provide protection via antioxidants from some types of cancer. Glycine is also used in the body to help construct normal DNA and RNA strands—the genetic material needed for proper cellular function and formation. It helps prevent the breakdown of muscle by boosting the body's levels of creatine. High concentrations of glycine are found not only in the muscles, but in the skin and other connective tissues as well. Almost ⅓ of collagen, which keeps the skin and connective tissue firm and flexible, is composed of glycine. Without glycine the body would not be able to repair damaged tissues; the skin would become slack as it succumbed to UV rays, oxidation, and free radical damage, and wounds would never heal.

Glycine is considered a glucogenic amino acid, which means it helps supply the body with glucose needed for energy. It helps regulate blood sugar levels, and thus glycine supplementation may be useful for treating symptoms characterized by low energy and fatigue, such as hypoglycemia, anemia, and Chronic Fatigue Syndrome (CFS). Glycine is essential for a healthy, normally functioning digestive system. It helps regulate the synthesis of the bile acid used to digest fats, and is included in many commercial gastric antacid agents.

Silymarin, another additive of the present invention, is an active extract from milk thistle comprised of a complex of 7 flavonolignans and polyphenols. The most active compound in slymarin is silibinin. It has been extensively used in patients with liver disease. Silymarin is a powerful antioxidant that protects liver cells (and other cells in the body and brain) from toxins. Sylimarin promotes liver cell protein synthesis and decreases the oxidation of glutathione. Silymarin is beneficial in a number of diseases involving liver disease, if administered in the early stages. Early research indicates that silymarin may also have anti-cancer properties.

The foregoing ingredients can be combined into an energy drink that promotes both health, mental acuity, and supplies with body with a temporary energy boost. In a preferred embodiment, the foregoing ingredients are combined into a drink having the following proportions:

### EXAMPLE No. 1

### Concentration per 1 oz./30 ml,

### Vitamin B1—2.1 mg,

### Vitamin B6—15 mg,

Vitamin B12—2.4 mcg (not mg),
Folic Acid—40 mcg (not mg),

### Taurine—50 mg,

### Glycine—100 mg,

Creatine 500 mg (0.5 grams),

### Silymarin—35 mg, and

### Caffeine—60 mg.

The caffeine preferably is within a range of 35 to 70 mg per ounce, and the creatine is preferably within the range of 400 to 600 mg per ounce. The ratio of creatine to caffeine is preferably in the range of approximately 10 to 1. In a preferred embodiment, the drink of the present invention is unsweetened, allowing it to be mixed with other drinks to suit the user's particular tastes. This allows the present invention to be used not only with juices or other sweetened beverages, but also with alcoholic beverages as a mixer. This latter use as an unflavored mixer with alcohol is presently unknown in today's energy drink market.

The foregoing description is intended to be exemplary and not limiting in any manner. Thus, nothing in the foregoing description should be read as limiting the present invention, and the descriptions and examples given are intended solely to provide a better understanding of the present invention. The scope and breadth of the present invention is properly determined from the appended claims, using the ordinary and customary meanings of the words used therein consistent with, but not limited by, the description of the invention.

I claim:

1. An energy beverage comprising:
caffeine; and
creatine.

2. The energy beverage of claim 1, wherein the concentration of caffeine is in the range of 35 mg to 70 mg per fluid ounce.

3. The energy beverage of claim 2, wherein the creatine concentration is in the range of 400 mg to 600 mg per fluid ounce.

4. The energy beverage of claim 1 wherein the ratio of caffeine to creatine is approximately ten to one.

5. The energy beverage of claim 1, wherein the beverage is unsweetened.

6. The energy beverage of claim 1, wherein the beverage is unflavored.

7. The energy beverage of claim 1, further comprising Vitamin B1.

8. The energy beverage of claim 7, further comprising Vitamin B6.

9. The energy beverage of claim 8, further comprising Vitamin B12.

10. The energy beverage of claim 1, further comprising folic acid.

11. The energy beverage of claim 1, further comprising taurine.

12. The energy beverage of claim 1, further comprising glycine.

13. The energy beverage of claim 1, further comprising silymarin.

14. The energy beverage of claim 1, further comprising alcohol.

15. The energy beverage of claim 1, further comprising taurine, glycine, and silymarin.
